(19) United States
(12) Patent Application Publication
(10) Pub. No.: US 2025/0349278 A1
(43) Pub. Date: Nov. 13, 2025

(54) AN OCCLUSION EFFECT MITIGATION DEVICE FOR AN EARPIECE

(71) Applicants: ÉCOLE DE TECHNOLOGIE SUPÉRIEURE, Montréal (CA); UNIVERSITE DU MANS, LE MANS (FR); INSTITUT DE RECHERCHE ROBERT-SAUVÉ EN SANTÉ ET SÉCURITÉ DU TRAVAIL, Montréal (CA)

(72) Inventors: Kévin CARILLO, Montréal (CA); Olivier DOUTRES, Montréal (CA); Franck SGARD, Montréal (CA); Olivier DAZEL, Le Mans (FR)

(21) Appl. No.: 18/855,525

(22) PCT Filed: Apr. 13, 2023
(86) PCT No.: PCT/CA2023/050504

§ 371 Date: Oct. 9, 2024

Related U.S. Application Data
(60) Provisional application No. 63/330,713, filed on Apr. 13, 2022.

(51) Int. Cl. G10K 11/172; A61F 11/08; A61F 11/14

(52) U.S. Cl. CPC G10K 11/172; A61F 11/085; A61F 11/14

(57) ABSTRACT

A hearing protection device that provides mitigation of an occlusion effect (OE) includes either an eartip for insertion into an ear canal or an over-the-ear cup having an internal member for covering an ear. The eartip and the internal member provide a medial surface. A plurality of resonator cavities are in fluid communication with the medial surface and each absorb an acoustic frequency to reduce, in combination, the occlusion effect.

### CROSS-REFERENCE TO RELATED APPLICATIONS

This patent application claims priority to U.S. provisional patent application 63/330,713, filed Apr. 13, 2022, the contents of which are hereby incorporated by reference.

### TECHNICAL FIELD

The present solution relates to the field of occlusion effect mitigation devices and more particularly to passive occlusion effect mitigation devices for an earpiece or an over-ear hearing protector.

### BACKGROUND

Among the many causes of hearing loss, occupational noise represents an important risk factor. Hearing protection devices such as earmuffs 100 and earplugs 102 are commonly used in noisy environments to protect workers from noise-induced hearing loss (NIHL), as shown in FIG. 1A. However, the lack of comfort associated with wearing hearing protectors strongly affects their use and thus their efficiency for preventing NIHL. Earplug comfort is a multidimensional construct that encompasses physical, functional, acoustical and psychological aspects. Regarding the acoustical dimension, the use of earplugs can induce with an occlusion effect. This phenomenon is described as an uncomfortable increased auditory perception of the bone-conducted part of one's own physiological noise (e.g., one's own voice, chewing, breathing, etc.) when the ear canal 106 entrance is covered or blocked, and is most significant at low frequencies, typically below one kilohertz (1 kHz).

The sensation of the occlusion effect is objectively associated with the augmentation of the acoustic pressure in the occluded ear canal 106, as shown in FIG. 1B, compared to the case where the ear canal is open, i.e., not obstructed such as by an earmuff 100 or earplug 102. It is usually quantified through the Occlusion Effect (OE) which is defined as the difference between the sound pressure level in Decibels at the eardrum in the occluded and open earcanals. An example of objective OE measured on participants is presented in graph 110 of FIG. 1C. Under a bone-conducted stimulation (e.g., vocal cords, bone-transducer, etc.), the ear canal wall 104 vibrates and generates an acoustic pressure in the ear canal cavity 106 which depends on the open or occluded state of the ear canal entrance. At low frequencies, the acoustic impedance (seen by the ear canal wall 104) of the occluded ear canal cavity is governed by the compressibility effect of the occluded volume and is significantly higher than the acoustic impedance of the open ear canal which is rather governed by its inertia effect. This change in character of the acoustic impedance of the ear canal seen by its wall 104 corresponds to the fundamental mechanism of the objective occlusion effect. Note that, as shown in FIG. 1B, the medial surface 108 of earplugs 102 also vibrates due to the bone-conducted stimulation and contributes together with the ear canal wall 104 to the acoustic pressure generated in the occluded ear canal 106. Compared to the contribution of the vibrating ear canal wall, the contribution of the vibrating earplug medial surface to the sound pressure level generated in the occluded ear canal is negligible at shallow insertion but predominant at deep insertion . . . .

Multiple solutions have been proposed in the past in order to occlude an ear canal while mitigating the occlusion effect. Solutions can be classified in two categories: active and passive solutions.

One passive method is to deeply insert the occlusion device in the ear canal. It can be used to reduce the occlusion effect induced by earplugs as well as hearing aids. This solution is based on the reduction of the vibrating ear canal wall area generating acoustic pressure in the occluded ear canal. However, the deep insertion can be responsible for mechanical discomfort due to the sensitivity of the ear canal wall in the bony part of the ear canal.

U.S. Pat. Nos. 8,848,939 and 9,539,147 to Keady and Hoshizaki disclose a method to reduce the occlusion effect with an inflating system to compress the ear canal walls. The expandable device is placed in the ear canal of a user, forming a sealed chamber. The pressure of the expandable device on the ear canal wall reduces the occlusion effect. However, the expandable device may exert an uncomfortable mechanical pressure on the ear canal wall. In addition, it requires a pump system to expand or contract the device.

Another passive solution is the use of vents 202 or open fittings 202 to drastically reduce the occlusion effect, in hearing aids 200, as shown in FIG. 2. This solution is based on the reduction of the acoustic impedance of the occluded ear canal (seen by the ear canal wall) compared to an acoustically rigid occlusion but comes at the cost of decreasing the hearing aid performance due to acoustic feedback, limited amplification gain and near zero suppression of ambient noise. Furthermore, this solution is not suitable for earplugs to ensure sufficient sound attenuation required for hearing protection purpose.

More recently, active systems reducing the occlusion effect of earplugs, hearing aids and earbuds have been developed based on the principle of destructive interference, as shown in the graph 300 of FIG. 3 and such as described in Design and Assessment of an Active Musician's Hearing Protection Device with Occlusion Effect Reduction by Bernier et al. (JAES 2021, 69, 618-631). The active systems use a loudspeaker that is placed inside the in-ear device to generate an anti-sound signal 302 that decreases the noise signal 304 level at low frequencies. These systems have a great potential to obtain a natural perception of one's own voice but can be subjected to the generation of distorted sounds when the sound pressure level in the occluded ear canal exceeds the maximum output level of the active noise cancelation system. In addition, these systems are likely to be more expensive than passive solutions.

Therefore, a passive solution to reduce the occlusion effect for improving the acoustic comfort of earplugs or earmuffs for users at shallow and medium insertion depths is thus required.

### SUMMARY

Noise-induced hearing loss (NIHL) is one of the most prevalent occupational conditions and occurs because of people exposed to excessive noise at their workplace. Hearing protection devices such as earmuffs and earplugs are used to protect workers from NIHL, however, the lack of comfort associated with those in-ear devices strongly affects their use. The use of earplugs is usually associated with the occlusion effect, an uncomfortable auditory perception of someone's own voice while talking, chewing, breathing, etc. The occlusion effect occurs when an object covers or fills the outer portion of the ear canal and is most significant at low frequencies (below 1 kHz). Active and passive solutions have been proposed in order to mitigate the occlusion effect. Active solutions are very expensive and require a battery and a unit to perform active noise control. The latter unit can be cumbersome for some users such as workers. Passive solutions, are commonly associated with physical discomfort, and reduced performance in the suppression of ambient noise.

In some embodiments, there is provided a hearing protection device providing mitigation of an occlusion effect (OE). The device may comprise for sound reduction, either an eartip for insertion into an ear canal or an over-the-ear cup, the eartip providing a medial surface and the ear cup having an internal member for covering an ear, the internal member providing a medial surface. The device may have a plurality of resonator cavities in fluid communication with the medial surface, the resonator cavities each absorbing an acoustic frequency and combining to reduce the occlusion effect.

The plurality of resonator cavities may be four in number and may be arranged as adjacent parallelepiped chambers. The resonator cavities may be configured to absorb at about 250 Hz, about 350 Hz, about 550 Hz and about 800 Hz.

The plurality of resonator cavities may be two in number and a first resonator cavity may be tuned for a range of about 225 Hz to 275 Hz with a second resonator cavity tuned to twice the frequency of the first resonator cavity, so as to subsequently reduce the Tonraum resonance and reduce said occlusion effect within a range of 100 Hz to 900 Hz.

The plurality of resonator cavities may be each in direct fluid communication with the medial surface.

One of the plurality of resonator cavities may be in direct fluid communication with the medial surface, and at least one other of the plurality of resonator cavities may be connected to the one of the plurality of resonator cavities in series.

When the device comprises the eartip, it may be configured to be supported by the eartip inserted in the ear canal. When the device comprises the eartip, it may be configured to be supported by a headband connected to the plurality of resonators.

When the device comprises the over-the-ear cup, the internal member may be made of foam. The internal member may support an array of the resonator cavities.

### BRIEF DESCRIPTION OF THE DRAWINGS

Further features and advantages of the present invention will become apparent from the following detailed description, taken in combination with the appended drawings, in which:

FIG. 1A presents commonly used prior art earmuffs and earplugs for hearing protection;

FIG. 1B presents a sectional view of an ear having the earplug of FIG. 1A inserted into the entrance of the ear canal;

FIG. 1C presents a graph showing an increase of the occlusion effect (OE) particularly at low frequencies and which characterizes the increase of the acoustic pressure within the occluded ear canal of FIG. 1B compared to the open one;

FIG. 2 presents a sectional view of an ear having a prior art vented earpiece inserted into the entrance of the ear canal in order to reduce the occlusion effect within the ear canal;

FIG. 3 presents a graph of signals in a prior art active noise cancelation system;

FIG. 4A presents an earpiece having an occlusion effect mitigation device according to one embodiment;

FIG. 4B presents an earmuff having multiple occlusion effect mitigation devices embedded inside the cup, according to one embodiment;

FIG. 4C presents alternate occlusion effect mitigation devices integrated in a simple earbud or attached with a headband, according to one embodiment;

FIG. 5A presents a schematic view of the occlusion effect mitigation device of FIG. 4 having four Helmholtz resonators positioned in parallel, according to one embodiment;

FIG. 5B presents a schematic view of the occlusion effect mitigation device of FIG. 4 having four Helmholtz resonators with chambers having respective foam layers inserted therein, according to one embodiment;

FIG. 5C presents the occlusion effect mitigation device of FIG. 5B as a foam layer is being inserted into the chamber of one of the four Helmholtz resonators, according to one embodiment;

FIG. 6 presents a schematic view of the occlusion effect mitigation device having four Helmholtz resonators positioned in series, according to one embodiment;

FIG. 7 presents the occlusion effect mitigation device of FIG. 4 and a corresponding three-dimensional schematic illustration of the Helmholtz resonators positioned in parallel within the occlusion effect mitigation device of FIG. 4, according to one embodiment;

FIG. 8 presents a frontal view and a side view of the Helmholtz resonators of the occlusion effect mitigation device of FIG. 4 with associated geometric parameters, according to one embodiment;

FIG. 9 presents a reflection coefficient graph at a medial surface of the occlusion effect mitigation device of FIG. 4, according to one embodiment;

FIG. 10 presents a normalized acoustic impedance graph at the medial surface of the occlusion effect mitigation device of FIG. 4, according to one embodiment;

FIG. 11 presents a graph with the occlusion effect induced by the device of FIG. 4 compared to a silicone earplug and a roll-down foam earplug, according to one embodiment; and

FIG. 12 presents a graph experimental occlusion effect measurements, according to one embodiment.

FIG. 13 presents a graph of the occlusion effect simulated using a theoretical model of the phenomenon induced by the occlusion effect mitigation device for two different optimization strategies minimizing the occlusion effect itself or the reflection coefficient R of the medial surface of the mitigation device.

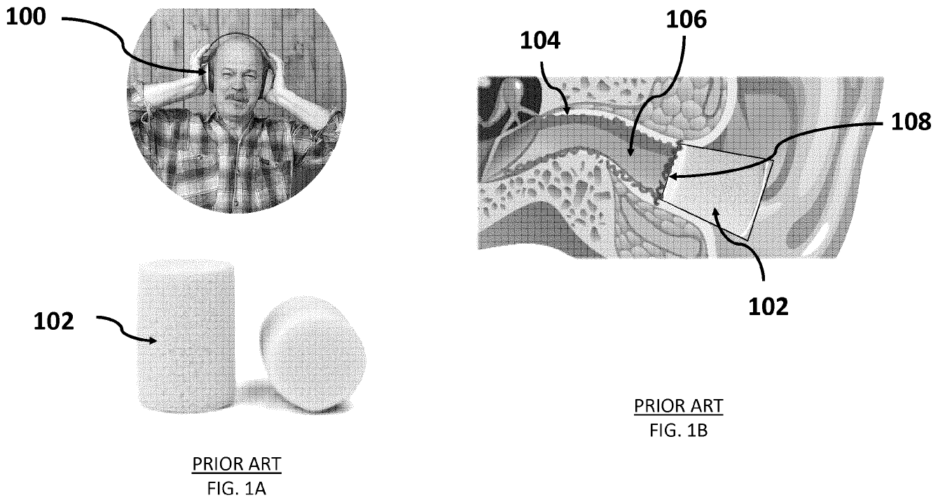

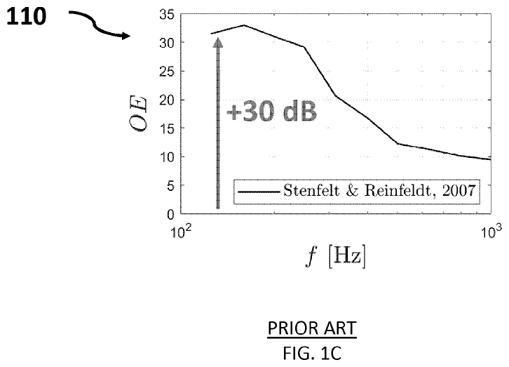

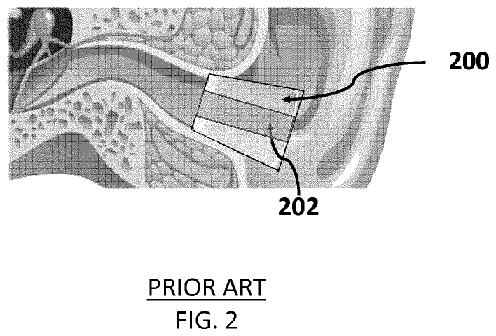

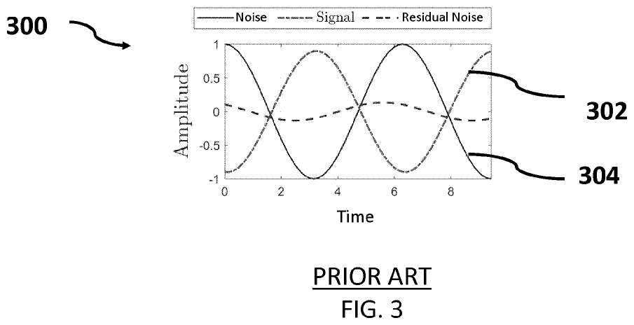

It will be noted that throughout the appended drawings, like features are identified by like reference numerals.

### DETAILED DESCRIPTION

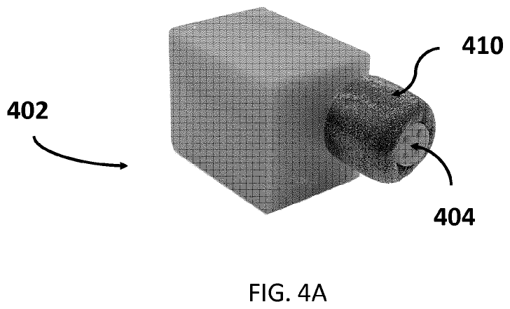

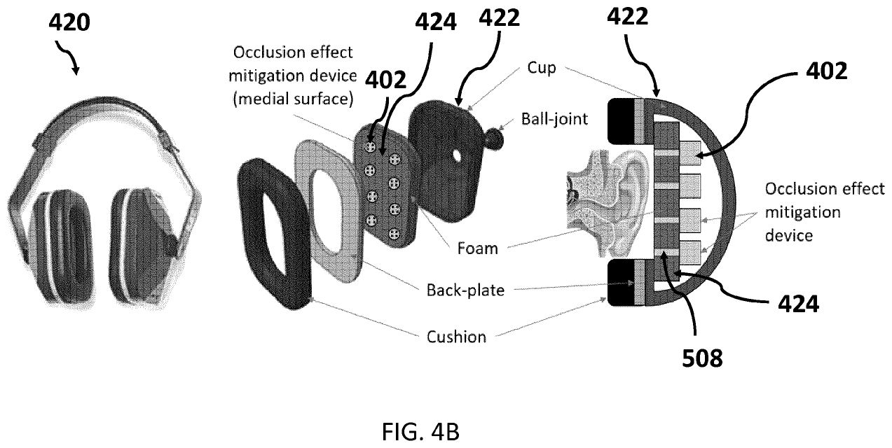

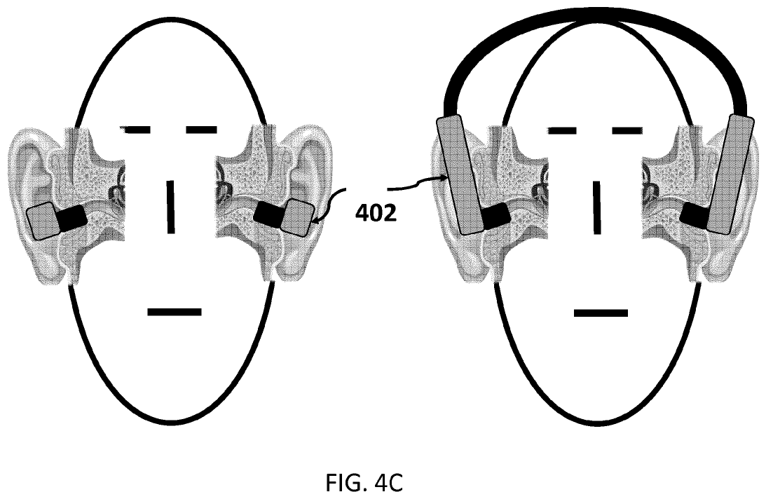

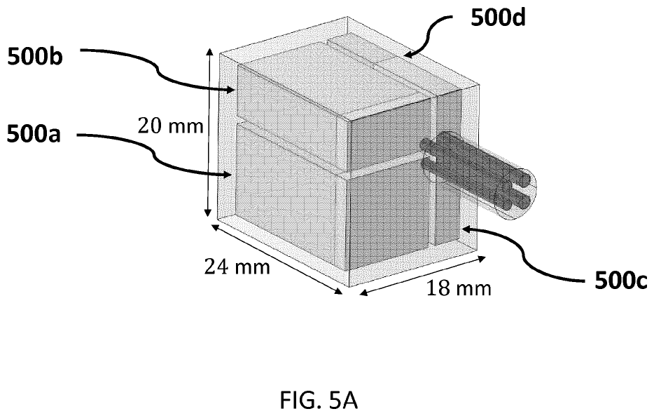

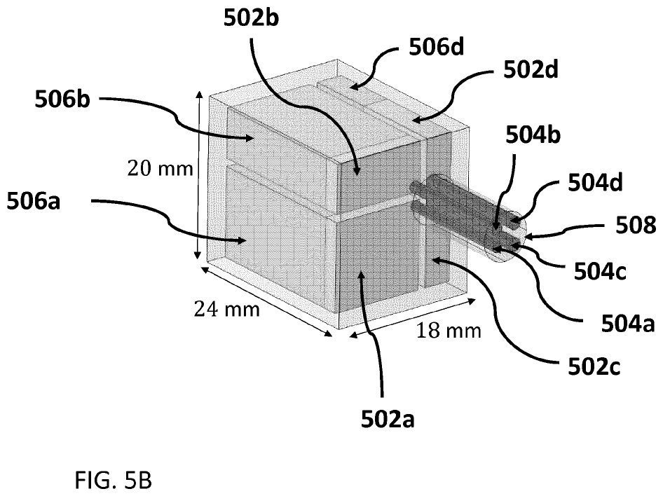

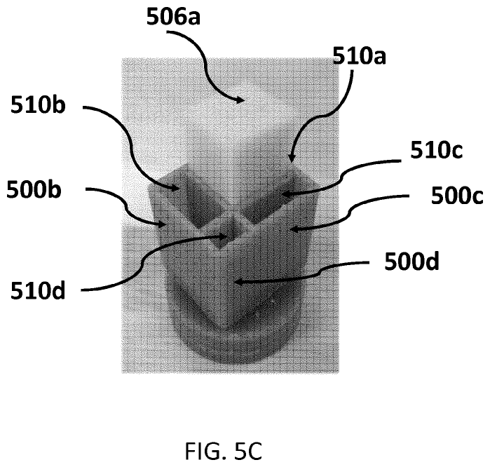

According to one embodiment, as presented in FIG. 4A, there is an occlusion effect (OE) mitigation device 402 dimensioned to seamlessly fit within an in-ear device such as an earplug, an earbud, a hearing aid, or an over-ear device such as an earmuff, headphone or headset. The OE mitigation device 402 has in some cases an eartip 410 adapted to be at least partially fittingly introduced within an ear canal in the case of an earplug, earbud or hearing aid. In the case of over-ear devices such as earmuffs, the eartip 410 is not required. The OE mitigation device 402 may further include a plurality of resonant systems (500a, 500b, 500c and 500d), such as Helmholtz resonators arranged in parallel and adapted to produce substantially the characteristic impedance of air at a medial surface 404 of the OE mitigation device 402 near the eartip 410 and thereby reduce the occlusion effect in a broad frequency range when the eartip 410 is introduced within the ear canal or within an over-ear device cavity, as concurrently shown in FIGS. 5A and 5B. Each resonant system (500a, 500b, 500c, 500d) is made of a neck (504a, 504b, 504c, 504d) and a cavity. The latter is partially filled with a sound absorbing material (506a, 506b, 506c, 506d), thus leaving an air gap (502a, 502b, 502c, 502d) in each resonator cavity. Each resonant system is configured to provide an effective balance between an energy leakage from a resonator (500a, 500b, 500c, 500d) to the environment and the energy dissipation within the resonator (500a, 500b, 500c, 500d) and together produce an acoustic absorption level that is similar to the acoustic absorption level that is provided by air at the medial surface 404. In the case of over-ear devices 420 such as the one presented in FIG. 4B, the cylindrical structure 508 gathering the resonators' necks can be inserted in a foam (or other suitable material) 424 that provides a medial surface inside a cup 422 of the over-ear device 420 or even embedded in the cup 422.

A skilled person will recognize that the number of resonators can differ from one embodiment to another depending on the frequency range in which a reduction occlusion effect or impedance is desired. It shall further be recognized that the target impedance of the mitigation device 402 at the medial surface 404 could be of lower or higher than the characteristic impedance of air, depending on the area of application.

Moreover, the sound absorbing material (506a, 506b, 506c, 506d) can be made of any material or combination of material adapted to absorb sound by way of visco-thermal dissipation mechanisms such as foam or fibrous materials or perforated/microperforated plates. It shall be recognized that the volume of respective sound absorbing material (506a, 506b, 506c, 506d) must be carefully selected according to the target impedance at the medial surface 404.

In addition and as further presented in FIGS. 5B and 5C, according to one embodiment, each cavity (510a, 510b, 510c, 510d) is only partially filled with a sound absorbing material (506a, 506b, 506c, 506d) and includes an air gap (502a, 502b, 502c, 502d).

As can be noticed in FIG. 5B, the necks (504a, 504b, 504c, 504d) are gathered in a thin cylindrical structure 508 that is insertable in the ear canal cartilaginous part. According to one embodiment, the cylindrical structure 508 is surrounded by a Comply® foam eartip 410 which can be adapted to various ear canal size and seal the entrance to ensure sound attenuation and maintain the OE mitigation device 402 in position, as shown in FIG. 4A. The cavities (510a, 510b, 510c, 510d) may be included in a parallelepipedic volume that measures approximately 7 cm³. Hence, the cavities (510a, 510b, 510c, 510d) of the OE mitigation device 402 could partially fit in the concha structure of the ear—whose volume measures around 4 cm³—and slightly protrude outside the pinna structure of the ear. As shown in FIG. 4C, the volume of the OE mitigation device 402 could be larger (for example, in order to target lower frequencies) and thus attached to a headband in order to be maintained in position.

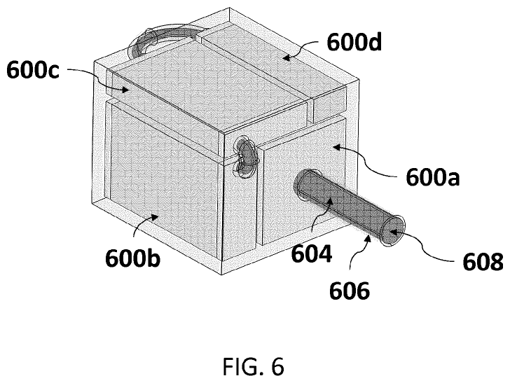

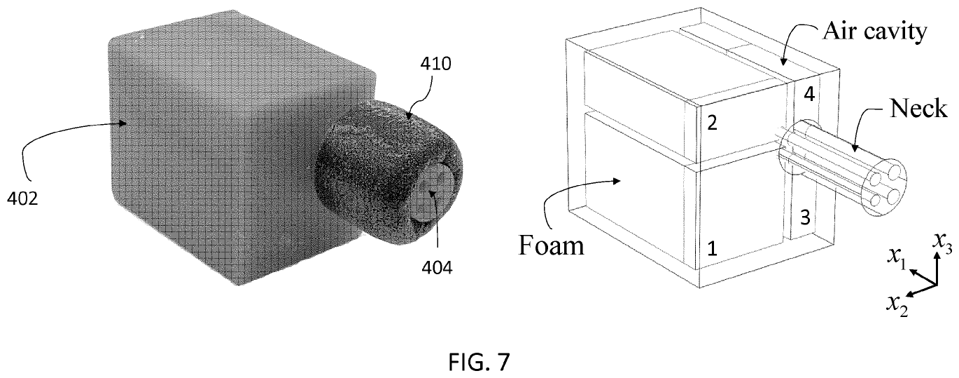

It shall be recognized that the shape, disposition and size of the cavities (510a, 510b, 510c, 510d) could differ from one embodiment to another. The geometry of the OE mitigation device 402 as a whole could differ such as to adapt to the shape of the pinna structure of the ear and to the ear canal geometry or to the over-ear device. For instance, according to one embodiment, the resonant systems (600a, 600b, 600c, and 600d) are arranged in series such, as shown in FIG. 6. In FIG. 6, a neck (604), the cylindrical structure (606) around it and the medial surface (608) are also shown. Compared to the parallel configuration of the OE mitigation device 402, the serial configuration only requires a single neck 604 to be inserted in the ear canal rather than all of them. Other necks connecting the cavities are semi-circular cylindrical ducts. The serial configuration allows to enlarge the radius of the necks, which increases the frequency bandwidth of the Helmholtz resonators' acoustic resonances that supports the broadband character of the acoustic properties of the medial surface of the OE mitigation device.

According to one optimization process, the geometry of the OE mitigation device 402 is determined in accordance with an evolutionary algorithm associated with a theoretical model of the reflection coefficient R of its medial surface 404. The cost function used in the optimization process is:

$$ε = {\int }_{{f}_{init}}^{{f}_{end}} {\vert R ( f ) \vert}^{2} df$$

where f is the frequency, to minimize the reflection coefficient in a broadband frequency range starting from fᵢₙₜ=200 Hz to f_{end}=900 Hz. The frequency range between 200 Hz and 900 Hz covers most of the frequencies where the occlusion effect is significant. Two geometrical parameters

$$( {e}_{cav}^{[ 1 ]} and {h}_{cav}^{[ 1 ]} )$$

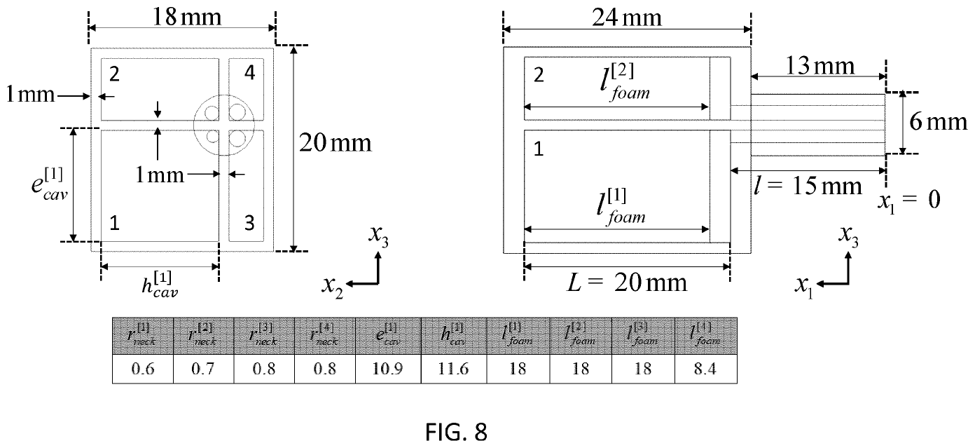

define the topology of the resonators and, for each resonator, the thickness of the foam layer (l_{foam}) and the radius of the neck (r_{neck}) are optimized. Geometrical parameters (in mm) resulting from the optimization process are summarized in FIG. 8, in which superscripts “1” to “4” refer to each resonators (500a, 500b, 500c and 500d).

Instead of minimizing the reflection coefficient R, the optimization process could also minimize the OE itself by using, for example, a theoretical model of the phenomenon induced by the OE mitigation device.

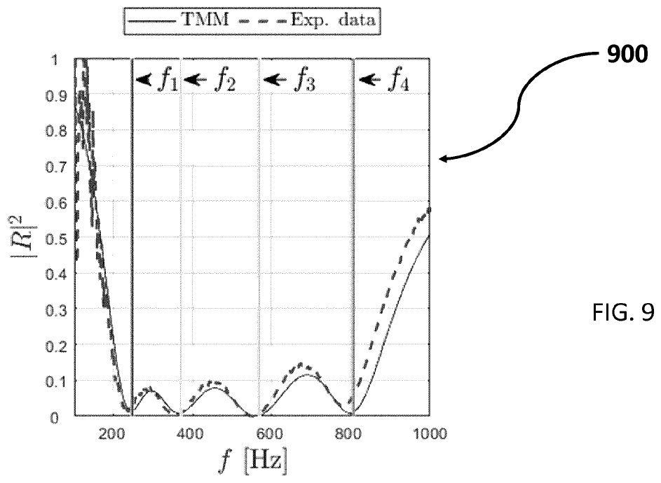

In FIG. 9, the continuous line represents the transfer matrix method (TMM) used to model and optimize the reflection coefficient R of the OE mitigation device 402 through the geometry of each resonator system (500a, 500b, 500c and 500d). The dotted line represents the reflection coefficient of the medial surface of the OE mitigation device obtained with experimental data. According to one embodiment and as presented in FIG. 9, the reflection coefficient graph 900 shows that the reflection coefficient of the OE mitigation device medial surface 404 is lower than 0.1 between 200 Hz and 900 Hz. This frequency range corresponds to that defined in the optimization process to cover the frequency region where the occlusion effect is most significant. Assuming negligible sound transmission through the structure of the OE mitigation device 402, the relation α=1−|R|², where a is the absorption coefficient, holds. Hence, the OE mitigation device medial surface 404 exhibits an acceptable broadband absorption between 200 and 900 Hz. In the graph 900, each vertical line indicates a frequency absorption peak of the OE mitigation device 402, (i.e., f₁=245 Hz, f₂=367 Hz, f₃=565 Hz, and f₄=803 Hz) each being produced by an associated resonator (500a, 500b, 500c and 500d).

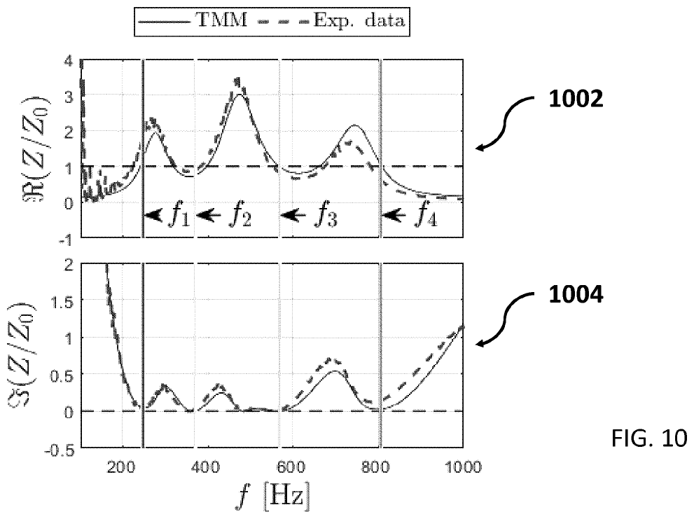

According to one embodiment, FIG. 10 shows that, at the absorption peaks of the OE mitigation device 402 (i.e., f₁-f₄), the real part of the normalized impedance—as shown in graph 1002—is close to 1 while its imaginary part—as shown in graph 1004—is almost 0. Hence, the acoustic impedance of the OE mitigation device medial surface 404 is mainly resistive and fulfils the conditions of impedance matching. However, below 200 Hz and above 900 Hz, the real part of the normalized impedance vanishes while its imaginary part increases. The acoustic impedance of the OE mitigation device medial surface 404 becomes thereby mainly reactive and departs from the impedance matching conditions.

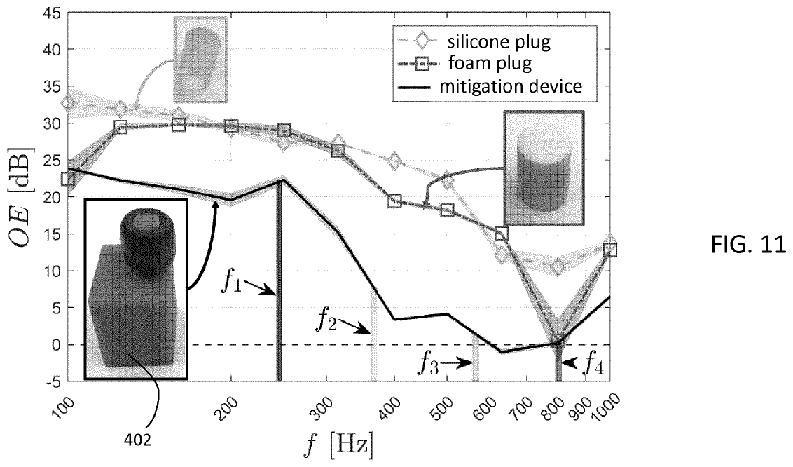

In FIG. 11, the occlusion effect induced by the OE mitigation device 402 is compared to a silicone earplug and a roll-down foam earplug inserted at the same insertion depth of an artificial outer ear, i.e., around 9 mm from the ear canal entrance. Results of the occlusion effect are presented in 3rd octave band in the frequency range 100 Hz-1 kHz. Vertical lines indicate absorption peaks frequencies (i.e., f₁-f₄) of the OE mitigation device 402.

The occlusion effect displayed in FIG. 11 for silicone and foam earplugs is shown to decrease with frequency from approximately 30 to 10 dB. This decrease is explained by the change in the character of the acoustic impedance of the ear canal seen by its wall between the mass-controlled open state and the compliance-controlled occluded state. According to FIG. 11, it can be noticed that the OE mitigation device 402 provides a significant broadband reduction of the occlusion effect compared to silicone and foam earplugs. This reduction reaches 15 to 20 dB (depending on which earplug is compared to the OE mitigation device 402) in the 3rd octave band centered at 400 Hz. Above this frequency, the occlusion effect induced by the OE mitigation device 402 remains lower than 5 dB. Regarding the silicone and the foam earplugs, their difference in occlusion effect is deemed to come from their difference in Poisson's ratio, which influences the vibro-acoustic contribution of their medial surface 404 to the sound pressure level generated in the occluded ear canal. For the OE mitigation device, the occlusion effect reduction rather comes from the acoustic properties of its medial surface 404.

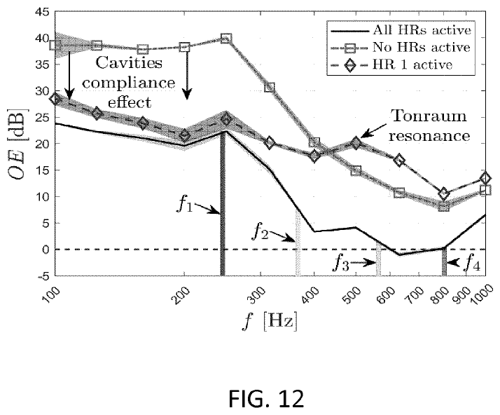

In order to examine the acoustic behavior of the OE mitigation device 402 for reducing the occlusion effect, FIG. 12 displays the experimental occlusion effect measured with all resonators (500a, 500b, 500c and 500d) active, no resonators (500a, 500b, 500c and 500d) active (neck of all resonators are obstructed) and only (HR #1) 500a active. When no resonators are active, the OE mitigation device medial surface 404 is acoustically rigid and its input acoustic impedance tends to infinity. Compared to the case with no resonators active, the OE mitigation device 402 with all resonators active provides a reduction of the occlusion effect from 15 to 5 dB between 100 Hz and 1 kHz and this reduction reaches almost 20 dB at 200 Hz. Between 200 and 900 Hz, this reduction is driven by the desired or substantially perfect broadband absorption of the OE mitigation device medial surface 404 whose input impedance approximately matches the characteristic impedance of air. Below 200 Hz, the perfect absorption behavior of the OE mitigation device 402 with all resonators active vanishes so the reduction of the occlusion effect it provides rather comes from the acoustic compliance of its chambers which decreases its input impedance depending on their total volume. This phenomenon is purely reactive and is similar to the reduction of the occlusion effect observed when using large earmuff.

When only resonator (HR #1) 500a only is active, FIG. 12 shows that the OE mitigation device 402 benefits from (i) the resonator volume for reducing the occlusion effect below 200 Hz and (ii) the perfect absorption ensured by its critical coupling at its resonance frequency around 250 Hz. Above this frequency, however, the acoustic absorption of the OE mitigation device 402 vanishes. In the 3rd octave band centered at 500 Hz and above, FIG. 15 shows that the occlusion effect induced by resonator (HR #1) 500a only is even larger than the occlusion effect produced when no resonators are active. This phenomenon is explained by the Tonraum acoustic resonance resulting from the coupling of resonator (HR #1) 500a to another finite volume, i.e., the ear canal cavity. When all resonators are active, the substantially perfect broadband absorption completely damps Tonraum resonances that could occur with resonators (HR #1) 500a, (HR #2) 500b and (HR #3) 500c and shifts out of the frequency range of interest the Tonraum resonance associated with resonator (HR #4) 500d where the occlusion effect is already low (typically above 1 kHz).

As will be appreciated, if only two resonators were to be chosen for the OE mitigation device, a first resonator tuned for the range of about 225 Hz to 275 Hz with a second resonator tuned to twice the frequency of the first resonator, so as to subsequently reduce the Tonraum resonance, could be an advantageous choice of only two resonators to limit the occlusion effect within the range of 100 Hz to 900 Hz.

By comparing FIGS. 11 and 12, it can be noticed that the OE mitigation device 402 with all resonators (500a, 500b, 500c and 500d) blocked induces a 5 to 10 dB higher occlusion effect compared to silicone and foam earplugs below 250 Hz. This increase in occlusion effect does not come from the acoustic properties of the OE mitigation device medial surface 404, which acts as an acoustically rigid surface when all resonators (500a, 500b, 500c and 500d) are blocked, similarly to silicone and foam earplugs. This increase rather originates from the mechanical behaviour of the OE mitigation device and its coupling with the ear canal wall and the ear canal cavity.

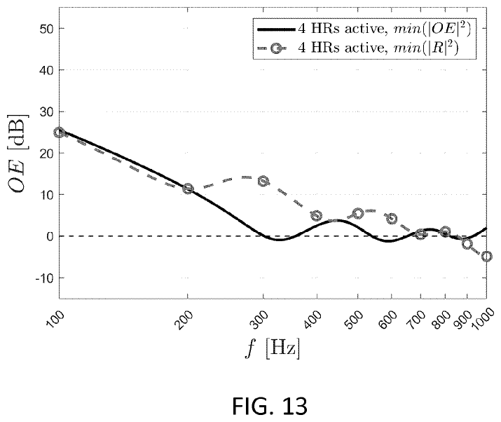

In FIG. 13, the optimization strategy minimizing the reflection coefficient R is compared to that minimizing the OE itself. The OE is computed using a theoretical model of the phenomenon induced by the OE mitigation device for the two optimization strategies. The optimization strategy minimizing the OE allows to further increase the OE reduction between 200 Hz and 700 Hz compared to the optimization strategy minimizing the reflection coefficient R. However, the strategy minimizing the OE requires the prior knowledge of the ear canal dimension and the ear canal wall vibration pattern corresponding to the ear canal in which the OE mitigation device is inserted.

In addition, earplugs that reduce the occlusion effect will also improve the protection for workers submitted to excessive noise level and whom are required to wear other hearing protection devices, such as earmuffs and/or helmets, in addition to their earplug. When hearing protectors are worn in combination (such as an earplug in combination with an earmuff), a non-negligible part of the sound pressure level at the eardrum origins from the sound transmitted through the head and body directly to the ear canal (also referred to as the outer ear bone conduction path). The contribution of this path could be greatly reduced by the use of the OE mitigation device since it reduces the acoustic impedance of the occluded ear canal (seen by its wall) and thus decreases the amplitude of the acoustic pressure that is generated in reaction of the ear canal walls vibration.

Compared to the use of vents, the proposed solution allows for reducing the objective occlusion effect while ensuring a sound attenuation adapted to hearing protection purposes or adapted to closed-fit hearing-aids and earphones such as earbuds.

1. A hearing protection device providing mitigation of an occlusion effect (OE), the device comprising:
for sound reduction, one of:
(i) an ear tip for insertion into an ear canal, the ear tip providing a medial surface having a side facing an ear canal; and
(ii) an over-the-ear cup having an internal member for covering an ear, the internal member providing a medial surface having a side facing an ear canal; and
a plurality of resonator cavities in fluid communication with said side facing an ear canal of said medial surface, said resonator cavities each arranged to absorb from said ear canal an acoustic frequency below 1000 Hz and combine to reduce said OE at frequencies below 1000 Hz.

2. The device as defined in claim 1, wherein said plurality of resonator cavities are four in number.

3. The device as defined in claim 2, wherein said plurality of resonator cavities are arranged as adjacent parallelepiped chambers.

4. The device as defined in claim 2, wherein said resonator cavities are configured to absorb at about 250 Hz, about 350 Hz, about 550 Hz and about 800 Hz.

5. The device as defined in claim 1, wherein said plurality of resonator cavities comprise a first resonator cavity tuned for a range of about 225 Hz to 275 Hz with a second resonator cavity tuned to twice the frequency of the first resonator cavity, so as to subsequently reduce the coupling between the first resonator cavity and the ear canal cavity so as to reduce said occlusion effect within a range of 100 Hz to 900 Hz.

6. The device as defined in claim 1, wherein said plurality of resonator cavities are each in direct fluid communication with said medial surface.

7. The device as defined in claim 1, wherein one of said plurality of resonator cavities is in direct fluid communication with said medial surface, and at least one other of said plurality of resonator cavities is connected to said one of said plurality of resonator cavities in series.

8. The device as defined in claim 1, comprising said ear tip.

9. The device as defined in claim 8, wherein said device is configured to be supported by said ear tip inserted in said ear canal.

10. The device as defined in claim 8, wherein said device is configured to be supported by a headband connected to said plurality of resonators.

11. The device as defined in claim 1, comprising said over-the-ear cup.

12. The device as defined in claim 11, wherein said internal member is made of foam.

13. The device as defined in claim 11, wherein said internal member supports an array of said resonator cavities.

14. The device as defined in claim 1, wherein said plurality of resonator cavities are partially filled with a sound absorbing material, leaving an air gap in each one of said plurality of resonator cavities, each one of said plurality of resonator cavities being configured to provide an effective balance between an energy leakage from each said one of said plurality of resonator cavities to the environment and an energy dissipation within each said one of said plurality of resonator cavities and together produce an acoustic absorption level that is similar to an acoustic absorption level that is provided by air at the medial surface.

15. The device as defined in claim 1, wherein said plurality of resonator cavities comprise a neck portion and a cavity portion, with the cavity portion preferably being partially or fully filled with sound absorbing material.

16. A hearing protection device providing mitigation of an occlusion effect (OE), the device comprising:
for occluding an ear canal of a wearer and for providing sound reduction of external noise for preventing hearing loss while working in noisy environments, one of:
(i) an ear tip for insertion into an ear canal, the ear tip providing a medial surface having a side facing an ear canal; and
(ii) an over-the-ear cup having an internal member for covering an ear, the internal member providing a medial surface having a side facing an ear canal; and
one or more resonator cavities in fluid communication with said side facing an ear canal of said medial surface, said resonator cavities each arranged to absorb from said ear canal an acoustic frequency below 1000 Hz and reduce said occlusion effect at frequencies below 1000 Hz.
